(19) United States
(12) Patent Application Publication
(10) Pub. No.: US 2010/0100734 A1
(43) Pub. Date: Apr. 22, 2010

(54) DIGITAL RADIOLOGY SYSTEM AND METHOD FOR IMPLEMENTING THE RADIOLOGY SYSTEM

(75) Inventor: Olivier Chastel, Voiron (FR)

Correspondence Address: DARBY & DARBY P.C., P.O. BOX 770, Church Street Station, New York, NY, 10008-0770, US

(73) Assignee: TRIXELL S.A.S., Moirans (FR)

(21) Appl. No.: 12/520,843

(22) PCT Filed: Dec. 18, 2007
(86) PCT No.: PCT/EP2007/064146

§ 371 Date: Jun. 22, 2009

(30) Foreign Application Priority Data
Dec. 21, 2006 (FR) 06 11211

(51) Int. Cl. G06F 15/16; H04L 9/32

(52) U.S. Cl. 713/168; 709/217

(57) ABSTRACT

The invention relates to a digital radiology system and a method of implementing the radiology system. The radiology system includes a mobile cassette and a fixed base station, the cassette including an X-ray image acquisition device to which the cassette is exposed, the system also including a communication interface between the cassette and the base station to enable transfer data such as the image between the cassette and the base station. The communication interface includes a removable wired link and a wireless link, both capable of transferring data and the system includes a circuit to deactivate the wireless link as soon as the wired link is set up. The method includes setting up as a priority a data interchange over the wireless link and switching the exchange over to the wired link as soon as the latter is set up.

This is a U.S. National Phase Application under 35 U.S.C. §371 of International Application no. PCT/EP2007/064146, filed Dec. 18, 2007, and claims benefit of French Patent Application No. 06 11211, filed Dec. 21, 2006, both of which are incorporated herein. The International Application was published in French on Jul. 10, 2008 as WO 2008/080837 under PCT Article 21 (2).

The invention relates to a digital radiology system and a method of implementing the radiology system. A digital radiology system essentially includes a number of elements such as an ionizing radiation source, such as, for example, an X-ray tube, for generating an X-ray, an X-ray detector making it possible to provide an image dependent on the received X-ray and a base station including an information processing system for synchronizing the X-ray tube and the detector and also for performing image processing operations such as presenting, to the operator, the image that is corrected of all the defects inherent in the detector and that is enhanced (for example by contour heightening processes). An object for which the image X is to be obtained is placed between the source and the detector. Such a system can be used in numerous applications such as, for example, medical radiology and non-destructive testing.

In the past, the radiology systems were bulky and largely immobile. The object had to be positioned relative to the system to obtain the desired image. With the appearance of solid-state detectors such as, for example, that described in the French patent application FR 2 605 166, the detector has become less bulky and it has been possible to move the detector relative to an object that remains fixed. For medical radiology digital detectors have been produced in the form of mobile cassettes that can then be placed in the immediate proximity of a patient for whom an image is required, when the state of health of the patient prevents him or her being moved to a radiology-dedicated room.

To ensure the high availability of the system, the cassette is linked to the base station by a wired link. However, this link limits the mobility of the cassette. It would be possible to imagine replacing the wired link with a wireless link. This has become possible with the emergence of recent wireless technologies and electronic components with very low consumption. However, in the radiology systems, wireless links have never been used because of the risks of link interference, for example electromagnetic, reducing the availability of the system. Moreover, should the battery included in the cassette become fully discharged, the radiology system remains unavailable until the battery is fully recharged (approximately 2 hours). Independent cassettes have also been implemented that make it possible to acquire and record a number of radiology images. These cassettes are periodically connected to the base station by a wired link to unload the images. This connection operation takes place after the image has been taken and does not therefore allow for the instantaneous use of the images.

The invention aims to overcome the problems cited above by proposing a radiology system that implements a wireless link and a wired link to connect the cassette to the base station.

To this end, the subject of the invention is a digital radiology system including a mobile cassette and a fixed base station, the cassette including image acquisition means dependent on an X-ray to which the cassette is exposed, the system also including means of communication between the cassette and the base station making it possible to transfer data such as the image between the cassette and the base station, characterized in that the communication means include a removable wired link and a wireless link both capable of transferring data, and in that the system includes means for deactivating the wireless link as soon as the wired link is set up.

Another subject of the invention is a method of implementing a digital radiology system including a mobile cassette and a fixed base station, the cassette including image acquisition means dependent on an X-ray to which the cassette is exposed, the system also including means of communication between the cassette and the base station making it possible to transfer data such as the image between the cassette and the base station, the communication means including a removable wired link and a wireless link both capable of transferring the data, characterized in that the method includes setting up a data interchange over the wireless link and switching the exchange over to the wired link as soon as the latter is set up.

Advantageously, one or more embodiments of the invention enables the base station to be paired with a cassette (for example, in the case of a replacement in the context of the after-sales service) in an environment where a number of wireless radiology systems coexist.

The invention will be better understood, and other benefits will become apparent, from reading the detailed description of an embodiment given by way of example, the description being illustrated by the appended drawing in which:

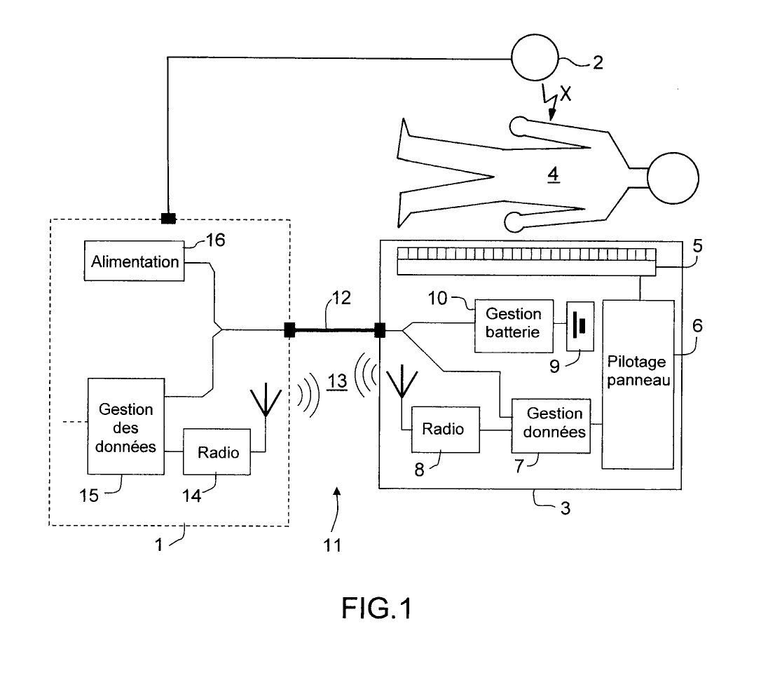

FIG. 1 diagrammatically represents a radiology system, including a base station and a mobile cassette, in accordance with one or more embodiments of the invention;

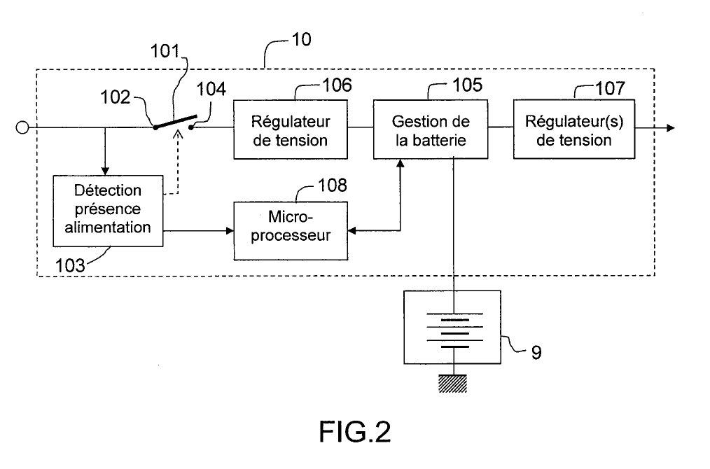

FIG. 2 represents in more detail various elements of the cassette power supply;

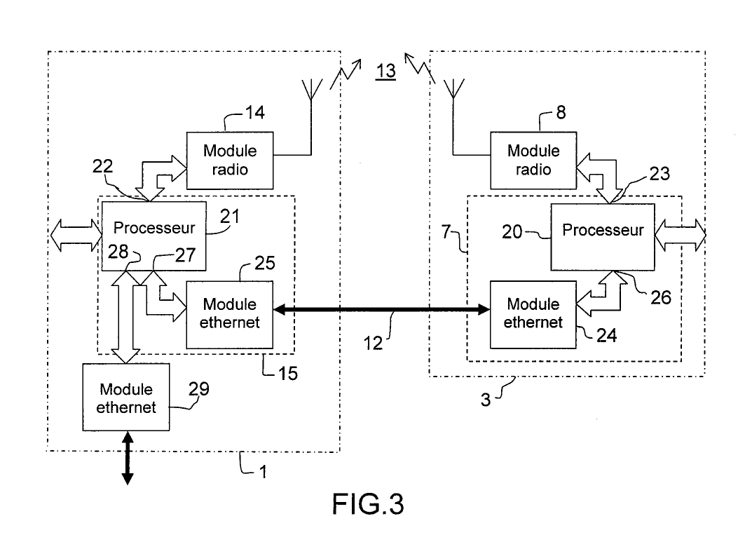

FIG. 3 represents in more detail an example of communication means between the cassette and the base station;

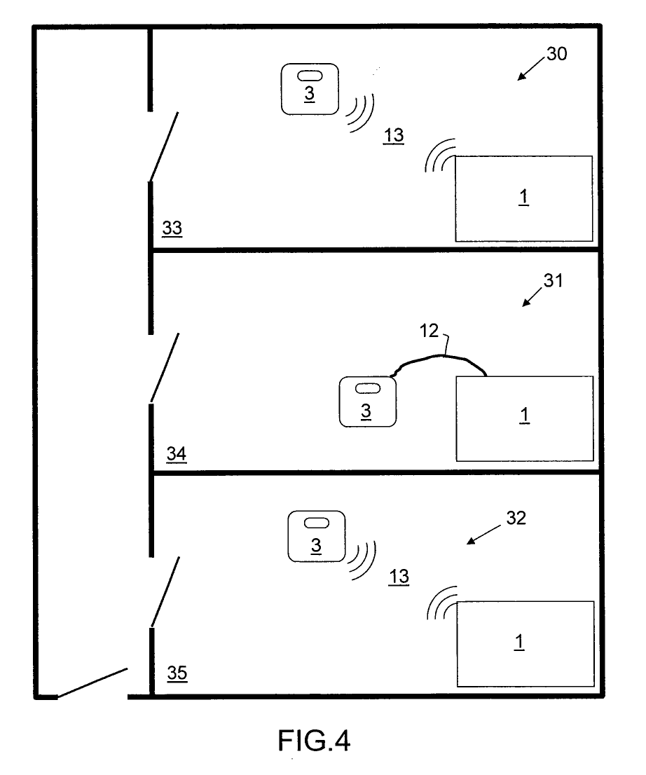

FIG. 4 represents the introduction of three radiology systems, each into a room, in order to better understand a method of pairing a base station with a cassette with no risk of confusion with the cassettes of the neighbouring radiology systems;

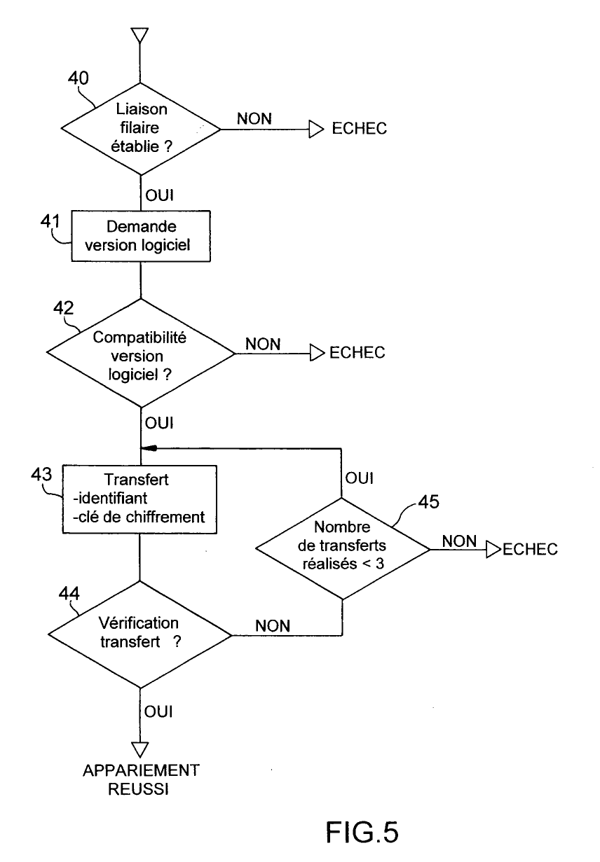

FIG. 5 is a flow diagram representing the pairing of the base station and the cassette.

In the interests of clarity, the same elements are given the same identifiers in the different figures.

FIG. 1 represents a radiology system intended for a medical use. The system comprises a fixed base station 1, an X-ray generator 2 and a radiation detector in the form of a mobile cassette 3. The cassette can be used to obtain an image of a patient 4 passed through by the X-radiation. The cassette 3 includes a digital detector implemented in the form of a flat panel 5 linked to a driver module 6 making it possible to read the image obtained by the flat panel 5 and digitize it through an analogue/digital converter. The mobile cassette 3 also includes a data management module 7, a radio module 8, a battery 9 and a battery management module 10.

The base station includes a radio module 14, a data management module 15 and a power supply 16.

Means 11 of communication between the cassette 3 and the base station 1 make it possible to transfer data such as the image between the cassette 3 and the base station 1. The data can circulate either from the base station 1 to the cassette 3, or from the cassette 3 to the base station 1. To the cassette 3, the data is, for example, control information for the flat panel 5, and to the base station 1, data includes, for example, the images produced by the flat panel 5.

The communication means comprise a removable wired link 12 and a wireless link 13. The two links 12 and 13 are both capable of transferring the data. The two radio modules 8 and 14 make it possible to exchange the data between the base station 1 and the cassette 3. The data management module 7 of the cassette 3 is used to switch the data received or originating from the driver module 6 to one of the links 12 or 13. Similarly, in the base station 1, the data management module 15 is used to switch the data received or originating from one of the links 12 or 13. The power supply 16 provides the necessary electrical energy for the various modules of the base station 1 and the cassette 3 to operate.

The power supply for the cassette 3 is fed via the wired link 12 or the battery 9. Advantageously, the system includes means for recharging the battery 9. More specifically, the battery management module 10 measures the charge of the battery 9 and initiates its recharging if necessary.

FIG. 2 gives a better understanding of how the battery management module 10 and the battery 9 operate. The battery management module 10 includes an electronic switch 101, a first terminal 102 of which receives electrical energy from the base station 1 via the wired link when the latter is set up. A power supply presence detection module 103 opens the switch 101 when the wired link is interrupted. The module 103 ensures the safety of the users of the cassette 3 and of the patient 4 when a connector of the cassette 3 receiving the wired link 12 is not connected. As a matter of fact, in the medical environment, the maximum currents that are allowed through a human body are very low, measuring just ten or so microamperes. To eliminate the risk of electrocution, the pins of the connector of the battery management module 10 are disconnected by means of the switch 101. A second terminal 104 of the switch 101 is connected to the core 105 of the module 10 via a voltage regulator 106 making it possible to smooth any fluctuations of the power supply voltage supplied by the wired link 12. These fluctuations are, for example, due to the length of the cable forming the wired link 12. The core 105 notably controls the charging current for the battery 9. A second voltage regulator 107 supplies the electrical power to the other components of the cassette 3, such as the flat panel 5. The battery management module 10 can, if necessary, include a number of second regulators 107 making it possible to supply various components of the cassette. A microprocessor 108 can handle the supervision of all of the battery management module 10. The microprocessor 8 can also inform the base station 1 as to the charge level of the battery 9 and its temperature.

According to one or more embodiments of the invention, the radiology system includes means for deactivating the wireless link 13 as soon as the wired link 12 is connected. The inventive method includes setting up as a priority a data interchange over the wireless link 13 and switching the exchange over to the wired link 12 when the latter is connected. The connection of the wired link 12 is, for example, detected by means of the power supply presence detection module 103. The electrical power supply for the cassette 3 operates as soon as the wired link 12 is set up.

FIG. 3 represents an exemplary embodiment of the communication means linking the base station 1 and the cassette 3. Each data management module 7 and 15 includes a processor, respectively 20 and 21, that are advantageously identical. It is possible, for example, to implement a reduced instruction set processor, well known from the literature as an RISC processor. This type of processor is ideal for systems operating in real time. Each processor has a number of ports making it possible to send and/or receive datastreams. A port 22 of the processor 21 is linked to the radio module 14 and a port 23 of the processor 20 is linked to the radio module 8. The modules 8 and 14 adapt the datastream sent and/or received according to a wireless transmission protocol such as, for example, that defined by the IEEE (Institute of Electrical and Electronics Engineers) standard 802.11 which is well known in the literature by the name WI-FI, standing for Wireless Fidelity. Other protocols can, of course, be used such as the Bluetooth protocol for example. This protocol is defined by major corporations in the telecommunications, computing and networking field such as Agere, Ericsson, IBM, Intel, Microsoft, Motorola, Nokia and Toshiba.

Each data management module 7 and 15 includes a network interface module, respectively 24 and 25. The module 24 is linked to a port 26 of the processor 20 and the module 25 is linked to a port 27 of the processor 21. The cable of the wired link 12 is connected between the modules 24 and 25. The modules 24 and 25 adapt the datastream sent and/or received according to a wired transmission protocol such as, for example, that defined by the IEEE 802.3 standard, well known as the Ethernet protocol. Other protocols are possible, such as RS 232 (standard defined by the Electronic Industries Association, based in the United States), or USB (Universal Serial Bus, a standard defined by an association of computer manufacturers: USB Implementers Forum, Inc, based in the United States). It is also possible to set up a protocol dedicated to the radiology system, called proprietary protocol. The wired link 12 can, for example, use an electrical or optical cable. The processors 20 and 21 can include other ports for data interchanges inside or outside the base station 1 and the cassette 3. As an example, a port 28 of the processor 21 has been represented linked to a network interface module 29. For the base station 1, the module 29 is, for example, used to transmit the image produced by the cassette 3 to storage and display means.

FIG. 4 represents the introduction of three radiology systems 30, 31 and 32, each into a room, respectively 33, 34 and 35. For the data interchange, the radiology systems 30 and 32 use their wireless link 13 and the radiology system 31 uses its wired link 12. The data interchange between one of the base stations 1 and the cassette 3 that is associated with it should be handled without risk of confusion with another base station 1 which would be installed in proximity, for example, in a neighbouring room. This risk of confusion appears with the use of the wireless link 13. In the case represented in FIG. 4, there is a risk of confusion appearing between the radiology systems used in the rooms 33 and 35. For this, in the data interchange at least over the wireless link 13, the base station 1 asks the cassette 3 for an identifier and allows the data interchange to continue only if it receives a response corresponding to an identifier stored in the base station 1 for the cassette 3. This check is, for example, defined by the IEEE 802.11 standard. It is possible to imagine pairing a base station 1 and a cassette 3 in the factory. However, this solution is very inflexible. This solution notably prevents the replacement of a cassette 3 on site. It is therefore useful to create a pairing procedure between a base station 1 and a cassette 3 on site.

Advantageously, the radiology system includes means for pairing the base station 1 and the cassette 3 when the wired link 12 is set up. The expression “setting up of the wired link” should be understood to mean the fact that the cable is connected and that data passes through the wired link 12. The inventive method includes pairing the base station 1 and the cassette 3 by means of the wired link 12. More specifically, the pairing is allowed only when the wired link 12 is set up and it is prohibited when the wireless link 13 is set up. In practice, it would be hazardous to perform a pairing between a base station 1 and a cassette 3 using the wireless link 13. In the case represented in FIG. 4, there would be a risk of associating the cassettes 3 of the rooms 33 and 35 with a single base station 1.

Advantageously, a cassette 3 and a base station 1 are automatically paired as soon as the wired link is set up. This procedure simplifies the operations of the radiology system. Nevertheless, this automatic function can present certain risks of involuntary pairings and it may be preferable for the pairing to take place only after a deliberate intervention on the part of a radiology system operator. In this case, it is even possible to pair the cassette 3 and the base station 1 only after a protected intervention on the part of a radiology system operator. In other words, the operator wishing to carry out the pairing should hold a particular authorization to perform this operation.

This operation may be embodied in the form of a physical key for example, or even in the form of a secret code that the operator should use to access the pairing procedure.

The pairing procedure includes, for example, an exchange of an identifier and possibly of a data encryption key, the encryption being implemented in data transfer over the wireless link 13 to ensure the confidentiality of the data exchanged.

The processors 20 and 21 of the cassette 3 and of the base station 1 each operate with software of a defined version. The versions of these software packages may evolve independently of each other. It is advantageous to check the compatibility of the software versions of the base station 1 and of the cassette 3 before carrying out the actual pairing operation.

A pairing method can be summarized by the successful sequencing of the operations illustrated in FIG. 5:
checking the setting up of the wired link 12. This operation is represented in the box 40 in the form of a test. If the wired link 12 is not set up, the pairing fails and the operator is notified thereof. If, on the other hand, the wired link 12 is set up, the method continues with the following operation:
checking the compatibility of the software versions of the base station 1 and of the cassette 3. To perform this check, the base station 1 interrogates the cassette 3 as to its software version. This interrogation is represented in the box 41. The compatibility is verified by the base station 1, for example, using a database. This compatibility check is represented in the box 42. In the event of incompatibility, the pairing fails and the operator is notified thereof. If, on the other hand, the compatibility is proven, the method continues with the following operation:
exchanging an identifier and a data encryption key, the encryption being implemented in the data transfer over the wireless link 13. The transfer of the identifier and of the encryption key from the base station 1 to the cassette 3 is represented in the box 43 and a check on the transfer is represented in the box 44. This check can include an interrogation by the base station 1 of the cassette 3 as to the identifier and the key that have been received that the base station 1 compares with those sent. If there is a difference between the elements sent and those received by return, up to two new transfer attempts can, for example, be made. The counting of the numbers of attempts is represented in box 45. Beyond three attempts, if the check is not correct, the pairing fails and the operator is notified thereof.

1. A digital radiology system comprising:
a mobile cassette, the mobile cassette comprising an X-ray image acquisition device that is exposed to X-rays;
a base station
a communication interface between the mobile cassette and the base station, to enable transfer of data wherein the communication interface comprises:
a removable wired link capable of transferring data; and
a wireless link capable of transferring data; and
wherein the system further includes a circuit to deactivate the wireless link after the wired link is set up.

2. The digital radiology system according to claim 1, wherein the mobile cassette further comprises:
a battery to power the mobile cassette; and
a battery management module to recharge the battery.

3. The digital radiology system according to claim 1, further comprising:
a circuit to pair the base station and the mobile cassette when the wired link is set up.

4. A method of implementing a digital radiology system, the system comprising:
a mobile cassette, the mobile cassette including an X-ray image acquisition device that is exposed to X-rays;
a base station
a communication interface between the mobile cassette and the base station, to enable transfer of data, wherein the communication interface comprises:
a removable wired link capable of transferring data; and
a wireless link capable of transferring data; and
wherein the method comprises the steps of:
setting up a data exchange over the wireless link; and
subsequent to the step of setting up, switching the data exchange over to the wired link as soon as the wired line is set up.

5. The method according to claim 4, wherein the method further comprises the step of supplying power to the mobile cassette via the wired link as soon as the wired link is set up.

6. The method according to claim 4, wherein the method further comprises the step of pairing the base station and the mobile cassette by use of the wired link.

7. The method according to claim 6, wherein the method further comprises the step of automatically pairing the mobile cassette and the base station as soon as the wired link is set up.

8. The method according to claim 6, wherein the method further comprises the step of pairing the mobile cassette and the base station after a deliberate intervention by a user.

9. The method according to claim 8, wherein the method further comprises the step of pairing the mobile cassette and the base station after a protected intervention by the user.

10. The method according to claim 6, wherein the base station and the mobile cassette each operate with a predetermined version of software, and the method further comprises the steps of:
checking a setting up of the wired link;
checking a compatibility of software versions of the base station and the mobile cassette;
exchanging an identifier and a data encryption key, the data encryption key being used in the transfer of data over the wireless link.
